(19) United States
(12) Patent Application Publication
(10) Pub. No.: US 2023/0154583 A1
(43) Pub. Date: May 18, 2023

(54) MEANS FOR CATEGORIZING AND TREATING DISORDERED SITUATIONS

(71) Applicant: Albert Burgess-Hull, Baltimore, MD (US)

(72) Inventor: Albert Burgess-Hull, Baltimore, MD (US)

(21) Appl. No.: 17/988,395

(22) Filed: Nov. 16, 2022

Related U.S. Application Data
(60) Provisional application No. 63/279,738, filed on Nov. 16, 2021.

(51) Int. Cl. G16H 15/00; G16H 50/70; G16H 50/20; G16H 50/30; G16H 40/67; G16B 20/00

(52) U.S. Cl. CPC G16H 15/00; G16H 50/70; G16H 50/20; G16H 50/30; G16H 40/67; G16B 20/00

(57) ABSTRACT

A method for treating a patient having a specified disorder includes the steps of: (a) compiling a treatment records database that includes patient longitudinal trajectories, (b) applying a plurality of clustering algorithms to identify a plurality of especially shaped, trajectory phenotypes for the disorder, (c) using a plurality of model selection techniques to determine the number and shape of the trajectory phenotypes, (d) compiling an intake database for said prior patients that includes background-characterizing factors for each of the patients, (d) using on the intake database analysis techniques to identify a means for classifying that predicts for a to-be-treated patient which of the trajectory phenotypes that is most similar to the real-time longitudinal trajectory that the to-be-treated patient will exhibit, and (e) identifying for the to-be-treated patient a treatment plan based on the to-be-treated patient's predicted trajectory phenotype.

### CROSS-REFERENCE TO RELATED APPLICATION

This Application claims the benefit of Provisional Patent Application No. 63/279,738, filed Nov. 16, 2021 by the present inventor. The teachings of this application are incorporated herein by reference to the extent that they do not conflict with the teaching herein.

### BACKGROUND OF THE INVENTION

### 1. Field of the Invention

This invention relates to a method and means for addressing or treating situations where disorder is present. More particularly, the present invention relates to the treatment of human disorders.

### 2. Description of the Related Art

A person can have a state of disorder that changes with the passage of time. For example, a person or patient can have a physiological disorder or the disorder can take the form of a clinical disease state of disease, and we speak of the state of the person's disease changing over time. The progression of a person's state of disorder through time can be denoted as the person's “longitudinal trajectory.” People or a group with similar longitudinal trajectories are said to define a “trajectory phenotype” for such a group and its members.

Knowledge of a patient's healthcare or medical longitudinal trajectory can be vital for decision-making regarding this patient's future, healthcare treatment. An accurate prediction of a patient's likely future, longitudinal trajectory would be of great value to those planning for this patient's healthcare treatment. For example, such knowledge could enable healthcare staff to select primary prevention measures for otherwise predicted, adverse occurrences in the patient's expected longitudinal trajectory.

Without a prediction for a patient's healthcare, longitudinal trajectory, healthcare staff often use a “wait and see” approach to medical care and treatment. For example, a “wait and see” approach can involve an individualized treatment plan which is revised only after observing changes in a patient's state; possibly resulting in extended treatment periods, wasted healthcare resources, and a higher risk for adverse outcomes.

Thus, there is a specific need for a method to generate a prediction for a patient's healthcare longitudinal trajectory that is both reliable and accurate. For other types of disorders that afflict people, there is also this need for a method to generate a prediction of a person's longitudinal trajectory with respect to such disorders.

The present invention seeks to provide such a method for a wide range of disorders.

### BRIEF DESCRIPTION OF THE DRAWINGS

FIG. 1 illustrates a user interface of the present invention that shows the generalized steps and elements that the present invention uses to help one address a situation in which the individual finds him/herself/themselves in a state of disorder.

FIG. 2 illustrates a user interface of the present invention that shows a plot of a real-time, longitudinal trajectory for one being treated and the trajectory phenotype appropriate for the subgroup into which this person has been classified.

FIG. 3 illustrates a user interface of the present invention that enables one being treated to document, record and store in a centralized, relational database, the magnitude, at various times, some of the variables that are used in quantifying the person's state of disorder.

FIGS. 4A-4E illustrate user interfaces of the present invention that present and communicate recommended treatment plans, individual participant characteristics, and participant performance information that can be useful in assessing one's response to his/her treatment.

FIG. 5 illustrates a user interface of the present invention that shows some of the typical patient information that one would expect to see in an Intake Database for those being treated for opioid-use disorders (OUDs).

FIG. 6 illustrates a user interface of the present invention that shows some of the typical patient information that one would expect to see in a Treatment Records Database for those being treated for OUDs.

FIG. 7 illustrates a user interface of the present invention that shows a plot of the trajectory phenotypes identified for various subgroups of patients that are being treated for OUDs.

FIG. 8 illustrates a user interface of the present invention that displays the membership size and some of the intake information for the patients in the four subgroups of FIG. 7.

FIG. 9 illustrates a user interface of the present invention that shows some of the differences between the disorder state behaviors for the patients in the four subgroups of FIG. 7.

FIG. 10 illustrates a user interface of the present invention that is for use in the OUD intake process.

FIG. 11 illustrates a user interface of the present invention that is for use by OUD patients to aid them in documenting and recording their craving levels at required times.

FIG. 12 illustrates a user interface of the present invention that is for use by a treatment professional in aiding the professional to document and record his/her treatment plan decisions.

### DESCRIPTION OF THE PREFERRED EMBODIMENT

Recognizing the need for a method of generating a prediction for a person's longitudinal trajectory with respect to a specified disorder, the present invention seeks to provide such a method or means; especially as it relates to a patient's healthcare longitudinal trajectory for different types of medical conditions.

Various aspects, advantages and alternative and preferred embodiments may be included in the following description of the present invention. All patents, patent applications, published articles and documents and other things referenced herein are hereby incorporated by this reference in their entirety and for all purposes. To the extent of any inconsistency or conflict in the definition or use of terms between any of the incorporated publications, documents or things and the present application, those of the present application shall prevail.

Before explaining at least one embodiment of the present invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

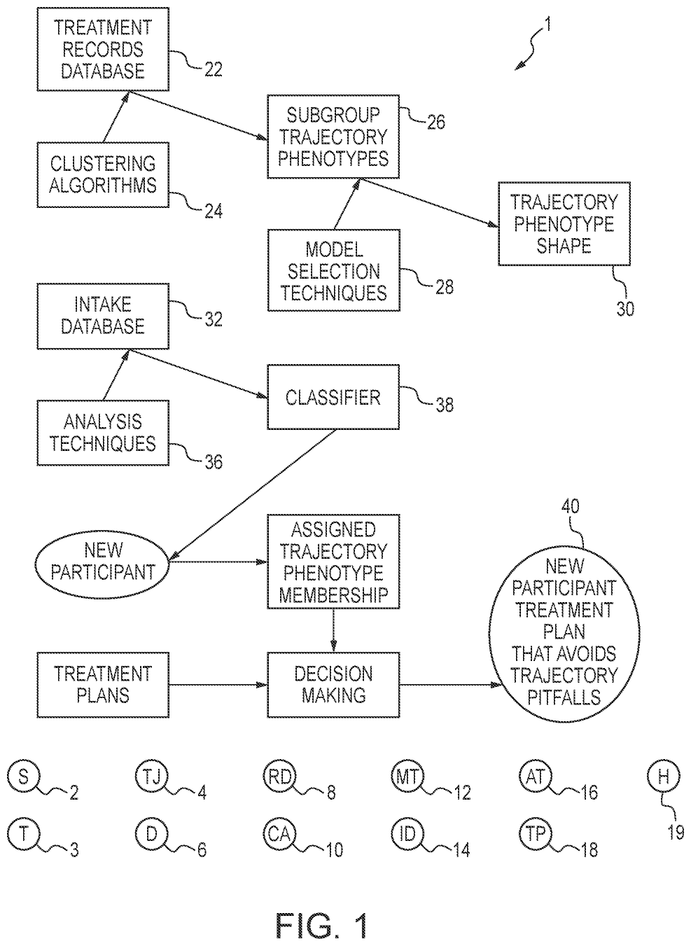

Illustrated in FIG. 1 is user interface of the present invention that shows the generalized steps and elements that the present invention 1 uses to help one address a situation in which the individual finds him/herself in a state of disorder (e.g., due to a health problem). This interface also includes various linkages (e.g., storage 2, treatment 3, trajectory 4, real-time data 6, treatment records database 8, clustering algorithms 10, modeling techniques 12, intake database 14, analysis techniques 16, treatment plan 18, help 19) that direct a user of the present invention to other user interfaces that are meant to help one in performing the various tasks of the present invention.

To be able to do this, the present invention makes a few assumptions about the person's state of disorder and the history of others that have been in similar disordered situations. These assumptions include: (1) that one's state or status of disorder at any time can be quantified in a numerical manner, (2) the means exist to track and quantify over time one's state of disorder, (3) a visualization of the progression over time of one's state of disorder can be displayed in a two-dimensional graph, where one's state of disorder at any time can be quantified and plotted on a vertical or y-axis, while the time at which this quantification occurs is plotted on a horizontal or longitudinal or x-axis; as previously noted, we refer to this as one's longitudinal trajectory 20 with respect to a specific disorder, (4) that there exist many other people who have previously found themselves in this same general state of disorder, and that each of these people have sought the help of a professional or attended a healthcare facility to address his/her/their disorder, and that this professional or clinic prescribed a course of action for addressing the disorder or remedying it (i.e., a course of treatment) and the impact of this treatment on the level of a person's disorder was quantified and recorded at various times in the progression of the disorder, and (5) that the professionals who previously treated these people, all of whom had this same general initial, state of disorder, conducted, for each of these people, pre-treatment interviews or data collection techniques that identified all of the possibly relevant factors that could have led to each person's eventual state of disorder when he, she or they initially sought treatment.

For disorders that meet the above assumptions, the present invention provides a method and system for addressing (e.g., treating over time) one's situation with respect to such a disorder by generally following the steps of: (1) compiling, from the known to exist treatment records of others who have previously dealt with this disorder, a treatment records database 22 of the longitudinal trajectories for each of these prior individuals receiving treatment, (2) applying a plurality of clustering algorithms 24 to this group of prior longitudinal trajectories to identify any trajectory phenotypes 26 for this disorder; (3) recognizing that these trajectory phenotypes are expressible in terms of a two-dimensional graph, using a plurality of model selection techniques 28 to determine the shape 30 and number of each of these identified trajectory phenotypes and noting what they predict the future status or state of disorder for one to which this trajectory phenotype applies, (4) compiling, from the nown to exist pre-treatment interview or other data records of others who have previously dealt with this disorder, an intake database 32 of the possibly relevant background-characterizing factors 34 that could have led to each person's state of disorder when he or she initially sought treatment, (5) developing, using this intake database and a plurality of statistical, mathematical and/or machine learning analysis techniques (i.e., “analysis techniques” 36), a classifier or means for classifying 38 that predicts, for a to-be-treated person seeking treatment for this disorder, into which of the previously identified trajectory phenotypes 26 this to-be-treated person will be classified; and (6) making a decision on how to address the to-be-treated person's disorder and identifying his/her initial treatment plan 40 based on the trajectory phenotype into which the person has been classified. This treatment plan will presumably be structured so as to help the person avoid the pitfalls that are predicted for him/her based on the trajectory phenotype 26 into which the person has been classified.

Once one or more persons have been in treatment for a period of time, another step in this method is undertaken: (7) their intake data and data on their treatment status over time (i.e., real-time data 42 for the patient) is collected and stored (including a participant's real-time, longitudinal trajectory). This collected data is then added to the previously collected data and the shapes of the trajectory phenotypes and the means for classifying new participants is reassessed (i.e., the classifier model is refit) in an attempt to improve the prediction capability of the initial classifier models.

The various analytical methods used by the present invention in accomplishing the above steps are significant and include: (a) clustering algorithms 24 that apply longitudinal or cross-sectional techniques (e.g., growth mixture modeling, k-means clustering, multi-layer neural networks), (b) mathematical and statistical model selection techniques 28, including the use of model fit indices, domain expertise, practical considerations, and other state-of-the-art model selection guidelines, and (c) statistical, mathematical and/or machine learning analysis techniques 36, including logistic regressions, support vector machines, neural networks, and boosted/bagged decision trees.

The present invention is especially useful for a professional who seeks to treat individuals with this disorder since it available to the professional in the form of a non-transitory, computable readable medium that has thereon program code which, with the professional's input, executes each of the above steps. For example, with respect to step (4) above, the program code of the present invention provides the means for conducting a pre-treatment or intake interview and records in the intake database 32 the outcomes of such an interview.

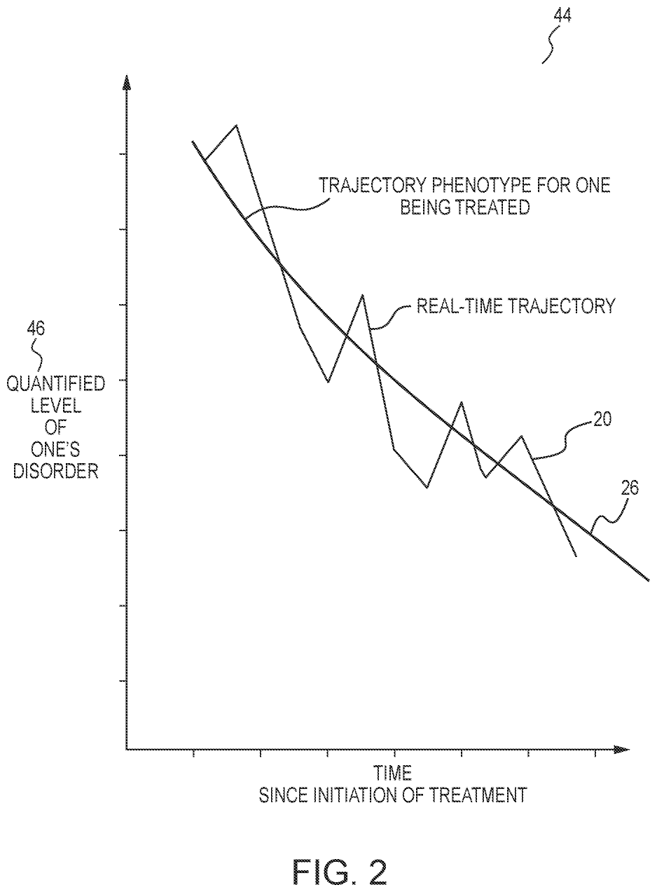

Additionally, this program code creates user interfaces 44 or graphical user interfaces (GUIs) that enhances the professional's ease in using the present invention. For example, it can compute and plot on the computer screen of a user of the present invention (e.g., a treatment-providing professional) the real-time, longitudinal trajectory of a being-treated person and any trajectory phenotype appropriate for the subgroup or group into which the person has been classified. See FIG. 2. Note also that this user interface and all the others described or shown herein are dynamic, since as more participant data is collected, these user interfaces are created and programmed in such a manner as to be updated.

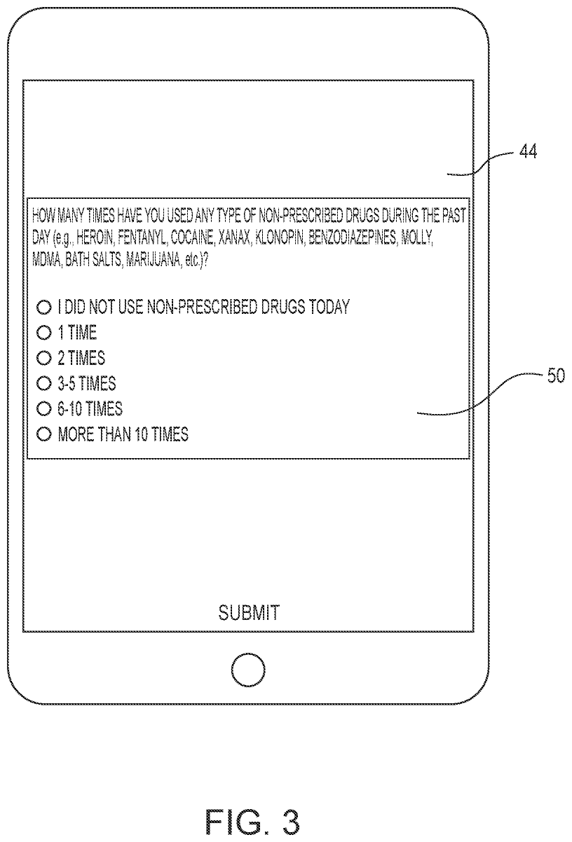

The program code of the present invention also has a portion that enables the person to use a unique user interface to document, record and store in a centralized, relational database, the magnitude, at various times, of the variables that are used in quantifying the person's state or status level of disorder 46, and/or those useful for predicting trajectory phenotypes. See FIG. 3.

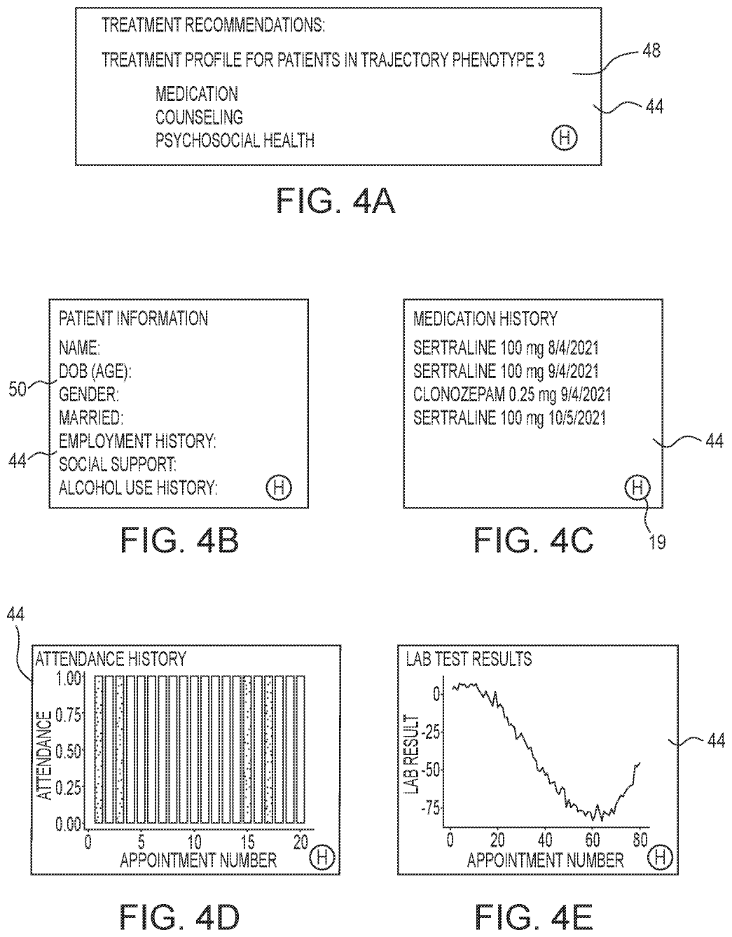

This program code can additionally create unique, user interfaces to present and communicate the results of the present invention, including: (a) recommended treatment plans 48—see FIG. 4A, (b) individual participant or in-treatment patient characteristics 50—see FIG. 4B, and (c) participant performance information 52 that can be useful in assessing one's response to his/her/their treatment—see FIGS. 4C-4E.

Note also that each of these user interfaces has a unique help button or linkage 19 where more detailed information can be found. This includes an optional expandable or pop-up panel when the help button is pushed or clicked that provides: (a) answers for commonly asked questions regarding the disorder, (b) probability estimates of the most likely trajectory phenotype, (c) how to interpret the personalized treatment recommendations, and (d) an input screen for feedback, comments, and suggestions.

The present invention can also be described as a system for use by treatment professionals that determines and communicates trajectory phenotype membership for to-be-treated persons and, if necessary, the updating of these membership classifications for people who are currently being treated. Several advantages of determining and then communicating the likely trajectory phenotype membership of new and current patients include that this information can be used by treatment professionals for: (a) the triaging of risk and the avoidance of predicted trajectory phenotype pitfalls, (b) understanding the expected responses to treatment, (c) better evaluating the disorder statuses of those being treated, and (d) improving the effectiveness of a professional's treatment plans.

While the above discussion has been directed towards using the present invention to address situations involving human disorders, it should be noted that the present invention is not limited only to these situations. For example, the present invention can also be used to address situations where the data collected to measure levels of disorder is not that which pertains to humans.

It is to be understood that the present invention can be applied to any situation that continues over a period of time and provides longitudinal data on a changing situation, and pre-event data (analogous to intake data) that can be used to predict membership in a specific longitudinal trajectory or trajectory phenotype. For example, longitudinal data may include data on insurance claims, financial products (e.g., stocks, bonds, derivatives, swaps), chemical processes, economic indicators, weather, animal performance data, and environmental or other dynamic processes.

To further enhance the disclosure of the present invention, it proves useful to demonstrate its application to a specific type of disorder. Consider the situation where the disorder in this hypothetical demonstration is a person's addiction to opioids, which is referred to as an opioid-use disorder (OUD).

Treatment with medication (buprenorphine or methadone) for opioid use disorder (MOUD) reliably reduces, but often does not eliminate, one's craving for opioids. Craving (i.e., the conscious, reportable urge to use drugs) is important to consider during MOUD for many reasons. First, for participants or patients in MOUD, craving is typically a distressing and intrusive experience, and thus inherently worth avoiding. Second, a patient's cravings over time has proven to be an effective, quantitative measure of the state or status of one's level of disorder 46.

A treatment records database 22 for OUD is easily created as there exist in the technical literature many examples of such databases that were created as a result of various clinical studies. For example, see Burgess-Hull et al., “Trajectories of craving during medication-assisted treatment for opioid-use disorder: Subtyping for early identification of higher risk;” published online at: https://www.sciencedirect.com/science/article/abs/pii/S0376871622000990, (2/18/22).

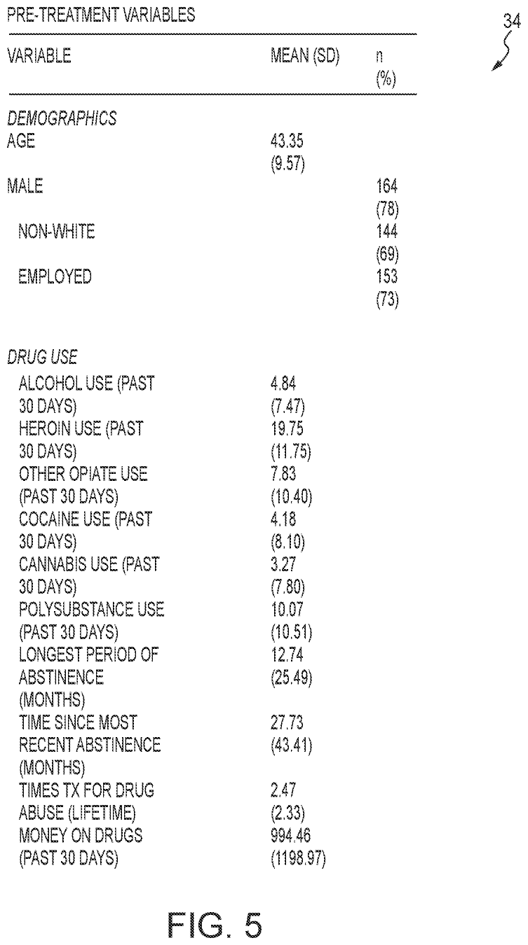

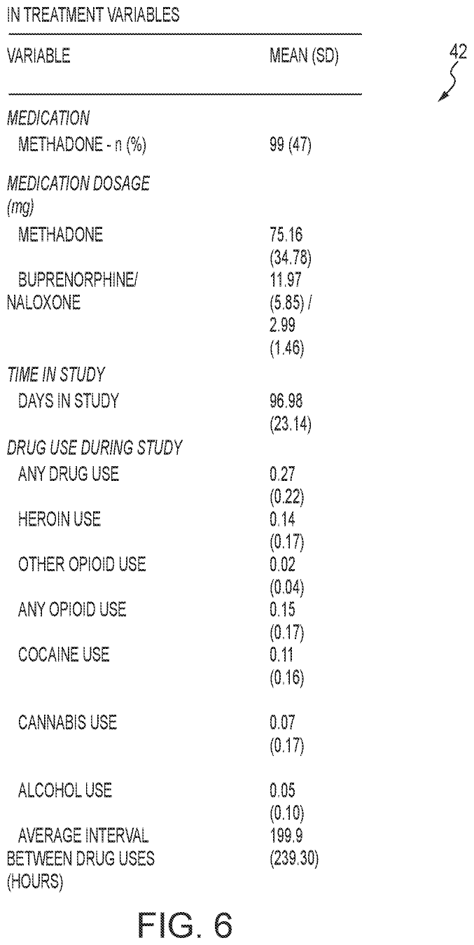

Hypothetically, all OUD participants would sign study-participation, consent forms and complete various intake activities (e.g., completing an Addiction Severity Index (ASI) interview and receive physical/psychosocial/biological assessments. The results of these intake activities would be used to create an intake database 32 for these participants. See FIGS. 5 and 6 that give a hypothetical, representative sample of some of the summarized data in such intake and post-intake databases.

Upon enrollment, each participant or patient would begin daily treatment with methadone or buprenorphine and attend required clinic sessions five to seven days a week and provide two to three weekly urine samples under observation. These urine samples would be screened for substances such as opioids, cocaine, amphetamines, PCP, benzodiazepines, and cannabinoids.

A participant's medication type would be determined by the participant's preference and clinical judgment of the study physician. Dosage would be optimized for each participant to minimize withdrawal symptoms and side effects and reduce illicit opioid use.

After two weeks of treatment, participants would receive a smartphone programmed by the present invention to emit three audible prompts per day at random times during each participant's normal waking hours. For each prompt, and since it was previously identified that this study would use one's level of craving as a measure of his/her opioid-use disorder, participants would be asked to rate their current opioid craving and stress level (1=“not at all” to 5=“extremely”), and make Ecological Momentary Assessment (EMA) entries, such as who they were with, and whether they had seen, been offered, or seen others using opioids, cocaine, cannabis, methamphetamine, tobacco, or alcohol in the last five minutes or since they got to their present location.

Such EMA could be quantified by noting when the patient felt ‘more stressed, overwhelmed, or anxious than usual’ (1=“not bad at all” to 10=“the worst you've ever felt”). For each event-contingent entry, participants would report if they were with others, whether they had seen others using drugs, and their current level of opioid craving.

To create a daily quantitative measure of the state or status of one's level of disorder 46, the study averages the random-prompt ratings of opioid craving within each day, generating a daily craving score. This data is stored in a treatment records database 22 and longitudinal trajectories 20 are created for each study participant.

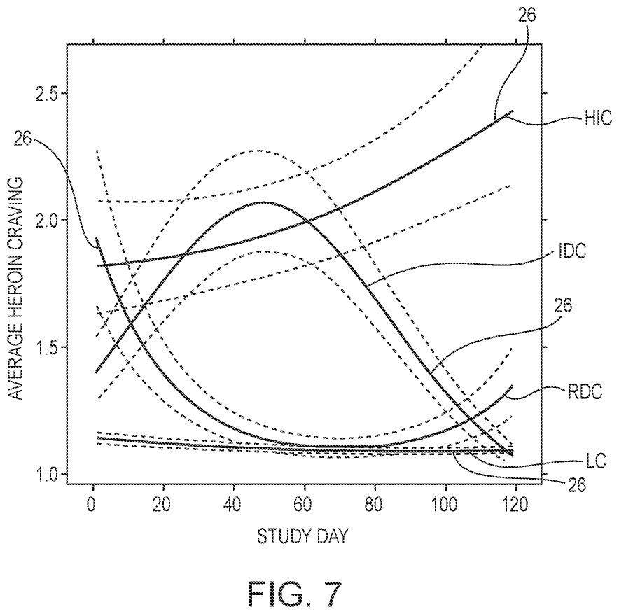

The present invention then applies various clustering algorithms 24 (e.g., growth machine modeling) to the collected data to identify any trajectory phenotypes 26 or trajectory subgroups for various groups of participants. The present invention would then apply a plurality of model selection techniques 28 to better determine the shapes 30 and number of the trajectories 20 in the participant sample. For this hypothetical, it was found that four trajectory subgroups were identified based on the participant craving trajectories. The hypothetical results of this effort are shown in FIG. 7 which illustrates the present invention's creation of a user interface 44 that is generated to show these results.

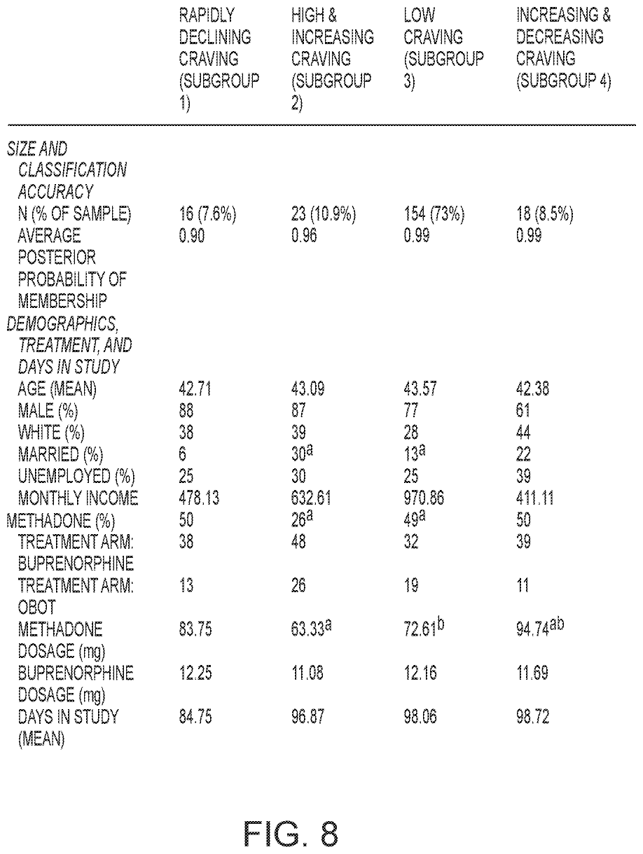

This user interface shows four identified subgroups; they are: a Low Craving trajectory (LC) subgroup that is characterized by consistently low levels of opioid craving during treatment, a Rapidly Declining Craving trajectory (RDC) trajectory subgroup that is characterized by high initial levels of opioid craving that decreased during treatment, a High and Increasing Craving trajectory (HIC) subgroup that is characterized by high initial levels of opioid craving that increased further during treatment, and an Increasing and Decreasing Craving trajectory (IDC) subgroup that is characterized by low initial levels of opioid craving that increased to a peak near the middle of treatment, then decreased. To provide some information regarding these subgroups, FIG. 8 illustrates the present invention's creation of another user interface 44 that is generated to display the membership size and some of the intake information for the participants in these various trajectory subgroups.

To determine whether the information collected during intake could be used to classify incoming participants into one of the four identified, craving-subgroups, the present invention would use various analysis techniques 12, including training a support vector machine (SVM) with a linear kernel using a subset of variables from the ASI.

Inverse-frequency class weights would be used to account for unbalanced subgroup sizes. Accuracy metrics are calculated using a leave-one-out cross-validation procedure, in which the SVM is trained with all datapoints except one which is used as a test set on which to make predictions. The procedure is repeated N times so that each datapoint serves as a test set once, where N is the number of datapoints.

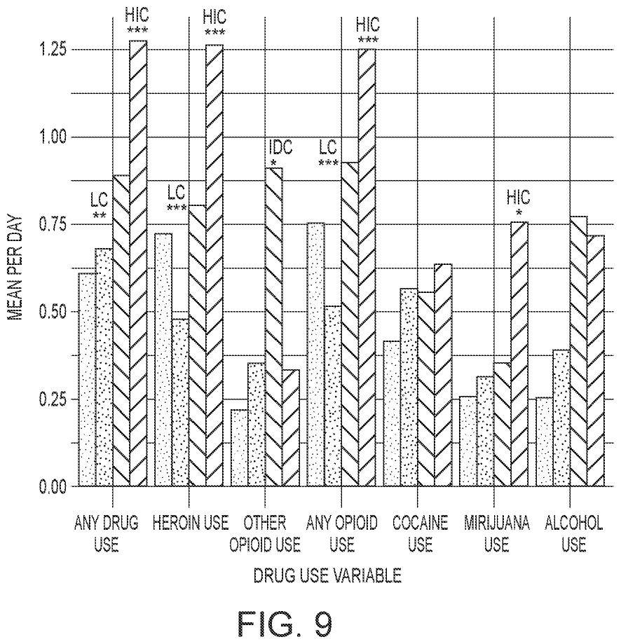

FIG. 9 illustrates a screen shot of another unique user interface 44 that is created by the program code of the present invention to show the differences between the four subgroups' intake information. These differences would be sufficient to enable one to create the required classifier that would allow one to assign a subgroup membership to a new participant based solely on his/her intake information.

With this demonstration now having shown how a classifier 38 can be developed to address OUDs, the remaining part of this demonstration needs only deal with the intake, classification and treatment of new patients seeking treatment. The software and program code of the present invention efficiently handles these tasks by its creation of many unique user interfaces that are designed to aid a treatment professional who chooses to use the present invention.

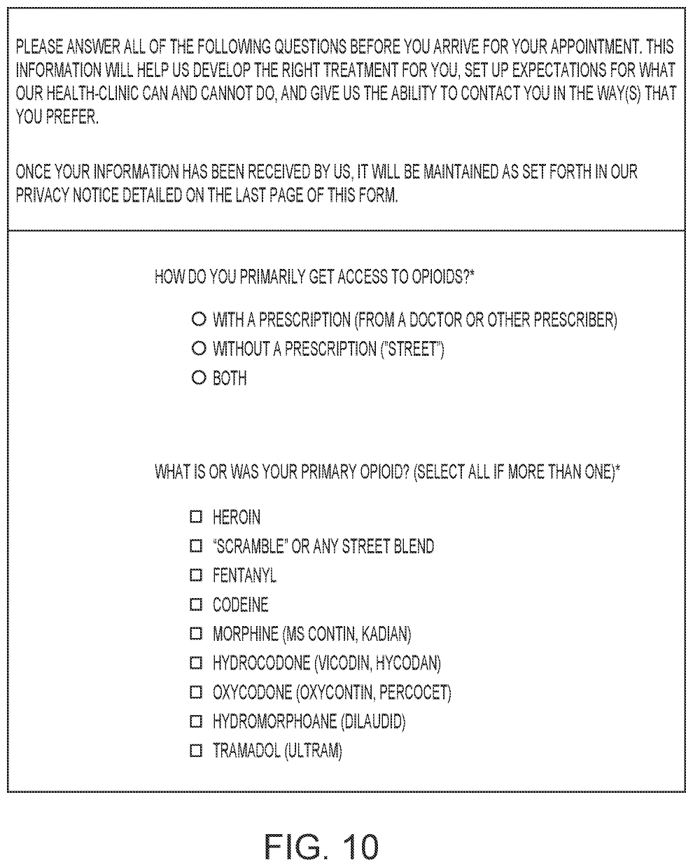

With respect to the OUD intake process, shown in FIG. 10 is a screen shot of a user interface that is created by the present invention for use in this process.

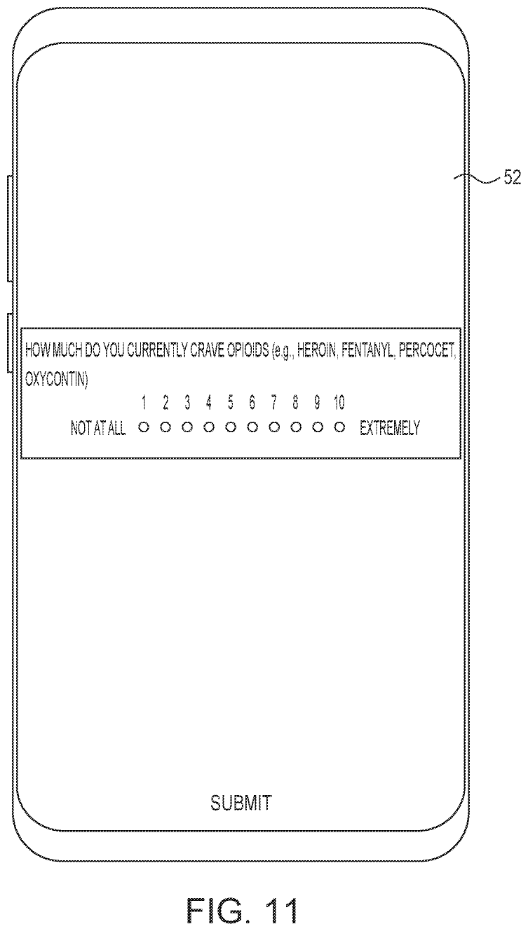

With respect to OUD treatment process, shown in FIG. 11 is a screen shot of a user interface with a real-time indicia and storage indicia that is created by the present invention for use by OUD patients to aid them in documenting, recording and storing their craving levels at required times.

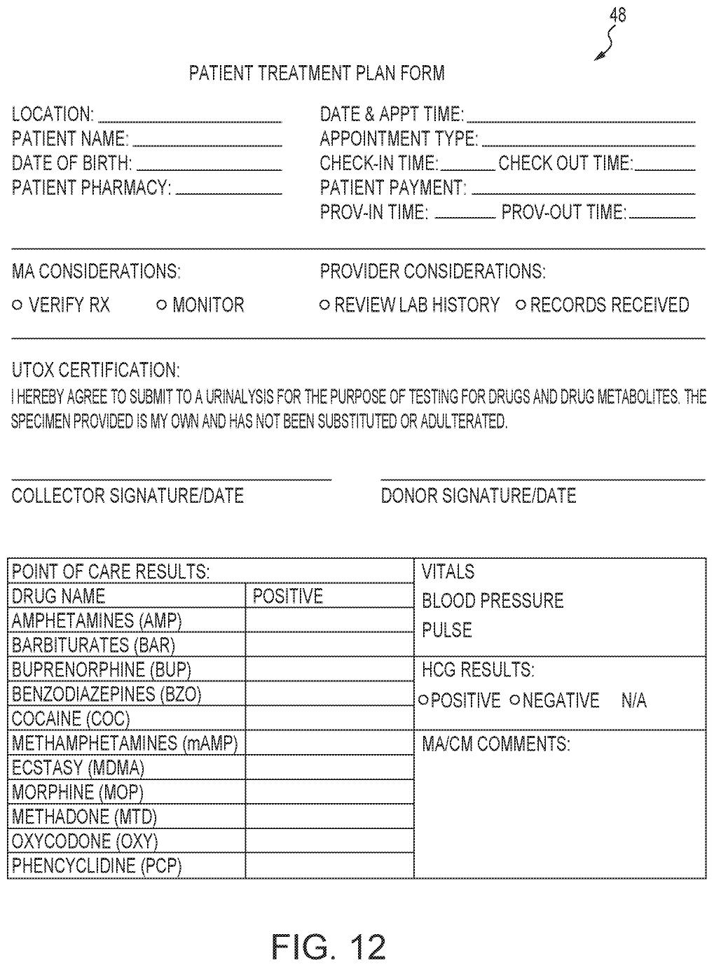

With respect to the task of deciding on a treatment plan for an OUD patient, shown in FIG. 12 is a screen shot of a user interface with a treatment indicia that is created by the present invention for use by a treatment professional in aiding the professional to document and record his/her/their treatment plan decisions.

The foregoing is considered as illustrative only of the principles of the present invention. Further, since numerous modifications and changes will readily occur to those skilled in the art, it is not desired to limit the invention to the exact construction and operation shown and described herein. Accordingly, all suitable modifications and equivalents may be resorted which fall within the scope of the invention that is hereinafter set forth in the claims to the invention.

I claim:

1. A method for treating a patient having a specified disorder, said method comprising the steps of:
compiling a treatment records database for prior patients who have been treated for said disorder, and including therein a longitudinal trajectory for each of said prior patients,
applying a plurality of clustering algorithms that are configured to analyze each of said longitudinal trajectories to identify a plurality of trajectory phenotypes for said disorder, wherein any of said trajectory phenotypes is mathematically expressible as a continuous line, with a distinctive shape, on a two-dimensional graph,
using a plurality of model selection techniques to determine said number and shape for each of said identified trajectory phenotypes,
compiling an intake database for said prior patients who have been treated for said disorder, and including therein a plurality of background-characterizing factors for each of said patients at the time of their entry into treatment,
using on said intake database a plurality of analysis techniques that are configured to identify a means for classifying, expressible in terms of said background-characterizing factors, that predicts, for a to-be-treated patient with said disorder who has provided at intake information on said background-characterizing factors, which of said plurality of trajectory phenotypes will have a shape that is most similar to the real-time longitudinal trajectory that said to-be-treated patient will exhibit, and classifying said to-be-treated patient to said trajectory phenotype, and
identifying for said to-be-treated patient a treatment plan based on said predicted trajectory phenotype of said to-be-treated patient.

2. The method as recited in claim 1, further comprising the step of:
once said to-be-treated patient has begun treatment and is now a new patient, collecting a plurality of real-time patient data from said new patient that quantifies, at specified times, the status level of said disorder for said new patient, and
adding said plurality of new patient data to said treatment records database, and redetermining said number and shape of said predicted trajectory phenotype for said new patient.

3. The method as recited in claim 1, further comprising the step of:
creating, on a display of a computing device of one using said method, a user interface that is configured to aid one in using said method.

4. The method as recited in claim 2, further comprising the step of:
creating, on a display of a computing device of one using said method, a user interface that is configured to aid one in using said method.

5. The method as recited in claim 1, wherein:
said specified disorder is an opioid-use disorder.

6. The method as recited in claim 2, wherein:
said specified disorder is an opioid-use disorder.

7. The method as recited in claim 3, wherein:
said specified disorder is an opioid-use disorder.

8. The method as recited in claim 7, wherein:
said user interface includes a storage linkage that allows said new patient to store both said real-time patient data and said plurality of background-characterizing factors for said new patient.

9. The method as recited in claim 7, wherein:
said user interface includes a treatment linkage that aids one treating a new patient to decide on a treatment plan for said new patient.

10. The method as recited in claim 7, wherein:
said user interface includes a trajectory linkage that displays both a real-time longitudinal trajectory and said predicted trajectory phenotype for said new patient.

11. A system that enable a treatment professional to treat a patient having a specified disorder, said system comprising:
a treatment records database for prior patients who have been treated for said disorder, and including therein a longitudinal trajectory for each of said prior patients,
a plurality of clustering algorithms that are configured to analyze each of said longitudinal trajectories to identify a plurality of trajectory phenotypes for said disorder, wherein any of said trajectory phenotypes is mathematically expressible as a continuous line, with a distinctive shape, on a two-dimensional graph,
a plurality of model selection techniques to determine said number and shape for each of said identified trajectory phenotypes,
an intake database for said prior patients who have been treated for said disorder, and including therein a plurality of background-characterizing factors for each of said patients at the time of their entry into treatment,
a plurality of analysis techniques that are configured to be used on said intake database to identify a means for classifying, expressible in terms of said background-characterizing factors, that predicts, for a to-be-treated patient with said disorder who has provided at intake information on said background-characterizing factors, which of said plurality of trajectory phenotypes will have a shape that is most similar to the real-time longitudinal trajectory that said to-be-treated patient will exhibit, and classifying said to-be-treated patient to said trajectory phenotype, and
a treatment plan for said to-be-treated patient that is based on said predicted trajectory phenotype of said to-be-treated patient.

12. The system as recited in claim 11, further comprising:
once said to-be-treated patient has begun treatment and is now a new patient, a plurality of real-time patient data from said new patient that quantifies, at specified times, the status of the level of disorder of said new patient.

13. The system as recited in claim 11, further comprising:
a user interface that is created on a display of a computing device of said treatment professional and configured to aid said treatment professional in using said method.

14. The system as recited in claim 12, further comprising:
a user interface that is created on a display of a computing device of said treatment professional and configured to aid said treatment professional in using said method.

15. The system as recited in claim 11, wherein:
said specified disorder is an opioid-use disorder.

16. The system as recited in claim 12, wherein:
said specified disorder is an opioid-use disorder.

17. The system as recited in claim 13, wherein:
said specified disorder is an opioid-use disorder.

18. The system as recited in claim 17, wherein:
said user interface includes a storage linkage that allows said new patient to collect and store both said real-time patient data and said plurality of background-characterizing factors for said new patient.

19. The system as recited in claim 17, wherein:
said user interface includes a treatment linkage that aids said treatment professional in deciding on a treatment plan for said new patient.

20. The system as recited in claim 17, wherein:
said user interface includes a trajectory linkage that displays both a real-time longitudinal trajectory and said predicted trajectory phenotype for said new patient.

21. A non-transitory, computer readable medium having program code recorded thereon, for execution on a computing device having a display, to enable a treatment professional to treat a patient having a specified disorder, said program code causing said computing device to perform the following method steps:
compiling a treatment records database for prior patients who have been treated for said disorder, and including therein a longitudinal trajectory for each of said prior patients,
applying a plurality of clustering algorithms that are configured to analyze each of said longitudinal trajectories to identify a plurality of trajectory phenotypes for said disorder, wherein any of said trajectory phenotypes is mathematically expressible as a continuous line, with a distinctive shape, on a two-dimensional graph,
using a plurality of model selection techniques to determine said number and shape for each of said identified trajectory phenotypes,
compiling an intake database for said prior patients who have been treated for said disorder, and including therein a plurality of background-characterizing factors for each of said patients at the time of their entry into treatment,
using on said intake database a plurality of analysis techniques that are configured to identify a means for classifying, expressible in terms of said background-characterizing factors, that predicts, for a to-be-treated patient with said disorder who has provided at intake information on said background-characterizing factors, which of said plurality of trajectory phenotypes will have a shape that is most similar to the real-time longitudinal trajectory that said to-be-treated patient will exhibit, and classifying said to-be-treated patient to said trajectory phenotype, and
identifying for said to-be-treated patient a treatment plan based on said predicted trajectory phenotype of said to-be-treated patient.

22. The non-transitory, computer readable medium having program code recorded thereon as recited in claim 21, said program code further causing said computing device to perform the method steps:
once said to-be-treated patient has begun treatment and is now a new patient, collecting real-time patient data from said new patient that quantifies, at specified times, the status of the level of disorder of said new patient, and
adding said new patient data to said treatment records database, and redetermining said number and shape of said predicted trajectory phenotype for said new patient.

23. The non-transitory, computer readable medium as recited in claim 21, said program code further causing said computing device to perform the method step:
creating on said display a user interface that is configured to aid one in using said method.

24. The non-transitory, computer readable medium as recited in claim 22, said program code further causing said computing device to perform the method step:
creating on said display a user interface that is configured to aid one in using said method.

25. The non-transitory, computer readable medium as recited in claim 21, wherein:
said specified disorder is an opioid-use disorder.

26. The non-transitory, computer readable medium as recited in claim 22, wherein:
said specified disorder is an opioid-use disorder.

27. The non-transitory, computer readable medium as recited in claim 23, wherein:
said specified disorder is an opioid-use disorder.

28. The non-transitory, computer readable medium as recited in claim 27, wherein:
said user interface includes a storage linkage that allows said new patient to collect and store both said real-time patient data and said plurality of background-characterizing factors for said new patient.

29. The non-transitory, computer readable medium as recited in claim 27, wherein:
said user interface includes a treatment linkage that aids said treatment professional in deciding on a treatment plan for said new patient.

30. The non-transitory, computer readable medium as recited in claim 27, wherein:
said user interface includes a trajectory linkage that displays both a real-time longitudinal trajectory and said predicted trajectory phenotype for said new patient.

31. A user interface for the display of the computing device of a treatment professional that enables said treatment professional to treat a patient having a specified disorder, said user interface comprising:
a treatment records database linkage that provides access to a treatment records database for prior patients who have been treated for said disorder, and including therein a longitudinal trajectory for each of said prior patients,
a clustering algorithms linkage that provides access to a plurality of clustering algorithms that are configured to analyze each of said longitudinal trajectories to identify a plurality of trajectory phenotypes for said disorder, wherein any of said trajectory phenotypes is mathematically expressible as a continuous line, with a distinctive shape, on a two-dimensional graph,
a modeling techniques linkage that provides access to a plurality of model selection techniques to determine said number and shape for each of said identified trajectory phenotypes,
an intake database linkage that provides access to an intake database for said prior patients who have been treated for said disorder, and including therein a plurality of background-characterizing factors for each of said patients at the time of their entry into treatment,
an analysis techniques linkage that provides access to a plurality of analysis techniques that are configured to be used on said intake database to identify a means for classifying, expressible in terms of said background-characterizing factors, that predicts, for a to-be-treated patient with said disorder who has provided at intake information on said background-characterizing factors, which of said plurality of trajectory phenotypes will have a shape that is most similar to the real-time longitudinal trajectory that said to-be-treated patient will exhibit, and classifying said to-be-treated patient to said trajectory phenotype, and
a treatment plan linkage that provides access to a treatment plan for said to-be-treated patient that is based on said predicted trajectory phenotype of said to-be-treated patient.

32. The user interface as recited in claim 31, further comprising:
once said to-be-treated patient has begun treatment and is now a new patient, a real-time data linkage that allows said new patient to collect a plurality of real-time patient data from said new patient that quantifies, at specified times, the status level of said disorder of said new patient.

33. The user interface as recited in claim 31, further comprising:
a help linkage that provides said treatment professional with access to help in utilizing said method.

34. The user interface as recited in claim 32, further comprising:
a help linkage that provides said treatment professional with access to help in utilizing said method.

35. The user interface as recited in claim 31, wherein:
said specified disorder is an opioid-use disorder.

36. The user interface as recited in claim 32, wherein:
said specified disorder is an opioid-use disorder.

37. The user interface as recited in claim 33, wherein:
said specified disorder is an opioid-use disorder.

38. The user interface as recited in claim 37, further comprising:
a storage linkage that allows said new patient to collect and store said plurality of background-characterizing factors for said new patient.

39. The user interface as recited in claim 37, further comprising:
a treatment linkage that aids said treatment professional in deciding on a treatment plan for said new patient.

40. The user interface as recited in claim 37, further comprising:
a trajectory linkage that displays both a real-time longitudinal trajectory and said predicted trajectory phenotype for said new patient.
